(19) United States
(12) Patent Application Publication
(10) Pub. No.: US 2025/0169895 A1
(43) Pub. Date: May 29, 2025

(54) SURGICAL ROBOT AND POWER SUPPLY PROTECTION CIRCUIT THEREOF

(71) Applicant: Precision Robotics (Hong Kong) Limited, Shatin, N.T. (HK)

(72) Inventors: Ping Lai Benny Lo, Shatin, N.T. (HK); Ka King Wong, Shatin, N.T. (HK)

(21) Appl. No.: 18/690,869

(22) PCT Filed: Aug. 17, 2023
(86) PCT No.: PCT/CN2023/113638

§ 371 Date: Mar. 11, 2024

Related U.S. Application Data
(60) Provisional application No. 63/398,550, filed on Aug. 17, 2022.

(51) Int. Cl. A61B 34/30

(52) U.S. Cl. CPC A61B 34/30

(57) ABSTRACT

The present disclosure provides a surgical robot and a power supply protection circuit thereof, which includes a main power supply module, a backup power supply module and at least one selection circuit. The selection circuit is configured to determine one of the main power supply module or the backup power supply module as power inputted according to input voltages detected from the main power supply module and the backup power supply module, and output power to supply power to the surgical robot. According to the present disclosure, the control of the automatic selection of power supply is realized by reasonably providing the selection circuit for voltage comparison, so that on one hand, the safe and normal use of the surgical robot can be ensured, and on the other hand, a battery of the backup power supply can be kept charged without being exhausted under the condition of normal power supply.

### CROSS-REFERENCE TO RELATED APPLICATIONS

This application is a National Stage Application under 35 U.S.C. § 371 of PCT Application No. PCT/CN2023/113638, filed Aug. 17, 2023 which claims priority to U.S. Provisional Patent Application No. 63/398,550, filed Aug. 17, 2022, the entire contents of which are incorporated by reference herein.

### TECHNICAL FIELD

The present disclosure relates to the technical field of a surgical robot, in particular to a surgical robot and a power supply protection circuit thereof.

### BACKGROUND

There are high requirements for the stability of power supply in the working process of surgical robots, but various objective factors usually determine that it is difficult to avoid some intermittent power supply stops. Uninterruptible power supply (UPS) is a power device that can continuously ensure the power supply to the load when the power is disconnected. However, due to its complicated control system and huge volume, it is difficult to be integrated and designed in a compact power supply system of the surgical robot to provide backup power supply.

### SUMMARY

A technical problem to be solved by the present disclosure is to provide a power supply protection circuit for a surgical robot for overcoming the above defects in the related art, which includes a main power supply module, a backup power supply module and at least one selection circuit.

The main power supply module and the backup power supply module are located at an input end of the selection circuit and are respectively configured to provide main power and backup power to the selection circuit.

The selection circuit is configured to determine one of the main power supply module and the backup power supply module as power inputted (to the surgical robot) according to input voltages detected from the main power supply module and the backup power supply module, and output power to supply power to the surgical robot.

This solution realizes the control of the automatic selection of power supply by reasonably providing the selection circuit for voltage comparison, which can ensure the stable and reliable output power supply and ensure the safe and normal use of the surgical robot.

Preferably, the selection circuit is configured to: select the main power supply module as the power inputted when a first input voltage corresponding to the main power supply module is not less than a preset voltage threshold; and select the backup power supply module as the power inputted when the first input voltage is less than the preset voltage threshold.

By defining a value of the first input voltage, this solution realizes the control of the automatic selection of power supply, which can ensure the stable and reliable output power supply and ensure the safe and normal use of the surgical robot.

Preferably, the preset voltage threshold is a maximum output voltage of the backup power supply module.

By setting the preset voltage threshold reasonably, this solution can realize that the power supply is always output by the main power supply module under normal conditions, so as to ensure the safe and normal use of the surgical robot.

Preferably, the selection circuit is configured to: select the main power supply module as the power inputted when a first input voltage corresponding to the main power supply module is not less than a second input voltage corresponding to the backup power supply module; and select the backup power supply module as the power inputted when the first input voltage is less than the second input voltage.

This solution realizes the automatic control and selection of power supply through numerical comparison and logical judgment of the first input voltage and the second input voltage, so as to ensure the safe and normal power supply of the surgical robot.

Preferably, the backup power supply module is configured to: output power to the selection circuit when it is used as the power inputted, and store power when the main power supply module is used as the power inputted.

According to the use characteristics of the backup power, this solution stores power for the backup power in time under the condition of normal power supply, thus ensuring the stable output of the backup power when needed.

Preferably, the backup power supply module includes a charging device and a rechargeable battery which are electrically connected with each other, and the charging device is configured to store power in the rechargeable battery when the main power supply module is used as the power inputted. In this solution, a backup power supply module is constructed by setting a charging device and a rechargeable battery, which can ensure the stability of power supply for the surgical robot by using the properties of the rechargeable battery.

Preferably, the power supply protection circuit for the surgical robot further includes a plurality of direct current (DC) to direct current (DC) converter. Input ends of the plurality of DC to DC converter are electrically connected to the rechargeable battery, and the output ends are electrically connected to a corresponding selection circuit.

Preferably, the power supply protection circuit includes at least two selection circuits with different output voltages. This solution realizes multi-input and multi-output power supply based on the setting of multiple selection circuits, which can meet the complicated and refined power supply requirements of the surgical robot and match more electrical devices and application scenarios.

The present disclosure further provides a power supply system for a surgical robot, which includes the above power supply protection circuit.

The present disclosure further provides a surgical robot, which includes the above power supply system.

The positive progress effect of the present disclosure is in that: the present disclosure provides a surgical robot and a power supply protection circuit thereof, and the control of the automatic selection of power supply is realized by reasonably providing the selection circuit for voltage comparison, so that on the one hand, the safe and normal use of the surgical robot can be ensured, and on the other hand, a battery of the backup power can be kept charged without being exhausted under the condition of normal power supply.

### BRIEF DESCRIPTION OF THE DRAWINGS

FIG. 1 is a schematic diagram of modules of a power supply protection circuit in Embodiment 1 of the present disclosure.

FIG. 2 is a schematic diagram of a voltage judgment logic of the power supply protection circuit in Embodiment 1 of the present disclosure.

FIG. 3 is a schematic diagram of a voltage judgment logic of the power supply protection circuit in Embodiment 1 of the present disclosure.

FIG. 4 is a schematic diagram of the power supply protection circuit module with a plurality of selection circuits in Embodiment 1 of the present disclosure.

FIG. 5 is a schematic diagram of modules of the power supply system for the surgical robot in Embodiment 2 of the present disclosure.

### DETAILED DESCRIPTION

In order to explain the technical solution of the embodiments of this specification more clearly, the accompanying drawings needed in the description of the embodiments will be briefly introduced below. Obviously, the accompanying drawings in the following description are only some examples or embodiments of this specification. For those skilled in the art, this specification can be applied to other similar situations according to these accompanying drawings without creative work. Unless it is obvious from the linguistic context or otherwise stated, the same reference signs in the accompanying drawings represent the same structure or operation.

As shown in this specification, the words “a”, “an”, “one” and/or “the” do not refer to the singular, but may also include the plural. Generally, the terms “include” and “contain” only imply the inclusion of clearly identified steps and elements, and these steps and elements do not constitute an exclusive list, and a method or device may also contain other steps or elements.

### Embodiment 1

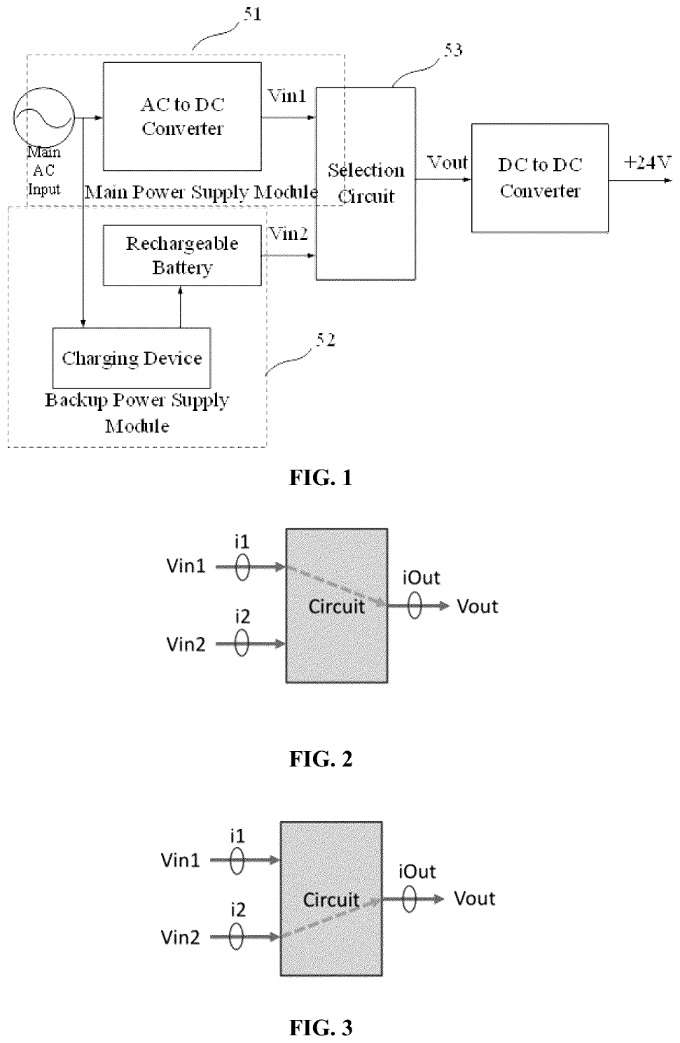

As shown in FIG. 1, this embodiment specifically provides a power supply protection circuit for a surgical robot, including a main power supply module 51, a backup power supply module 52 and at least one selection circuit 53. The selection circuit 53 may be provided on a backup power control board 500.

The main power supply module 51 and the backup power supply module 52 are located at an input end of the selection circuit 53, and are respectively configured to provide main power and backup power to the selection circuit 53. It can be understood that in this embodiment, the AC input is used as the main power supply module 51, and a plurality of DC to DC converter are provided in the power supply protection circuit, the input ends of which are electrically connected with the rechargeable battery, and the output ends of which are electrically connected with the corresponding selection circuit 53, and the main AC input can be converted into DC power for use.

The selection circuit 53 is configured to determine one of the main power supply module 51 and the backup power supply module 52 as power inputted according to input voltages detected from the main power supply module 51 and the backup power supply module 52, and output power to supply power to the surgical robot.

As a preferred embodiment, the selection circuit 53 is configured to: select the main power supply module 51 as the power inputted when a first input voltage corresponding to the main power supply module 51 is not less than a preset voltage threshold; and select the backup power supply module 52 as the power inputted when the first input voltage is less than the preset voltage threshold. Preferably, the preset voltage threshold is a maximum output voltage of the backup power supply module 52. That is, when the preset voltage threshold is set to this level, the system can be guaranteed to be powered by the main power supply module under normal conditions. In this embodiment, the control of the automatic selection of power supply is realized by reasonably providing the selection circuit for voltage comparison, which can ensure stable and reliable output power supply and ensure the safe and normal use of the surgical robot.

As a preferred embodiment, the selection circuit 53 is configured to: select the main power supply module 51 as the power inputted when a first input voltage corresponding to the main power supply module 51 is not less than a second input voltage corresponding to the backup power supply module 52; and select the backup power supply module 52 as the power inputted when the first input voltage is less than the second input voltage. This embodiment realizes the automatic control and selection of power supply through numerical comparison and logical judgment of the first input voltage and the second input voltage, so as to ensure the safe and normal power supply of the surgical robot.

Specifically, referring to the comparison and selection mechanism shown in FIG. 2 and FIG. 3, when an input of Vin1 exists, an input of Vin2 will stop, and Vin1 will be used as the power. If Vin1 is not inputted or it is lower than Vin2, Vin2 (such as a rechargeable battery) will be the power of the system. The voltage of Vin1 can be set higher than the maximum voltage of Vin2, then Vin1 is normally a power inputted with higher priority, that is, this power will supply power to the system. If the AC power is disconnected (e.g., the AC power wire is unplugged), so that the input of Vin1 does not exist, then Vin2 will continue to supply power in the system, that is, at this time, Vin2 (such as the rechargeable battery) will be used as the power of the system.

In this embodiment, by logically comparing and controlling the input voltages corresponding to the main power supply module 51 and the backup power supply module 52, it is possible to reasonably define the currently suitable input power to ensure power supply.

As a preferred embodiment, the backup power supply module 52 is configured to output power to the selection circuit 53 when it is used as power inputted, and store power when the main power supply module 51 is used as power inputted. The backup power supply module 52 includes a charging device and a rechargeable battery which are electrically connected with each other. The charging device is configured to store power in the rechargeable battery when the main power supply module 51 is used as the power inputted.

In this embodiment, by using the properties of the charging device and the rechargeable batteries in the power supply module 52, synchronous power storage can be performed when the system is in normal operation, that is, power is supplied by the main power supply module 51, so that the backup power can be kept sufficient, and the backup power can be provided in time when necessary.

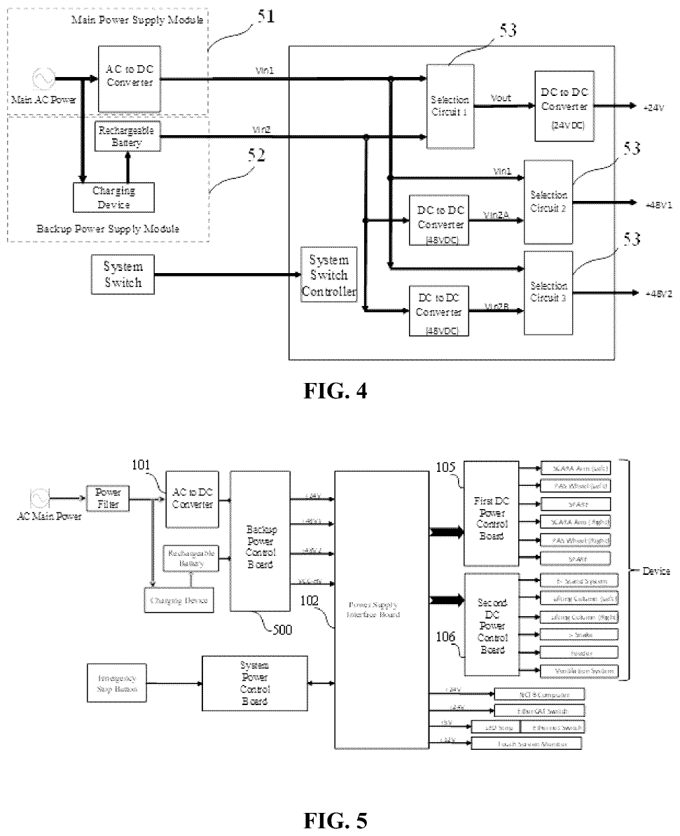

Preferably, the power supply protection circuit includes at least two selection circuits 53 with different output voltages. As shown in FIG. 4, multiple selection circuits can be provided in the backup power control board 500 to perform logic processing and output power supply synchronously, so as to form more judgment combinations and output modes, thus meeting the requirements of different electrical device.

The power supply protection circuit for the surgical robot in this embodiment realizes the control of the automatic selection of power supply by reasonably providing the selection circuit for voltage comparison, so that on the one hand, the safe and normal use of the surgical robot can be ensured, and on the other hand, a battery of the backup power can be kept charged without being exhausted under the condition of normal power supply.

### Embodiment 2

As shown in FIG. 5, this embodiment specifically provides a power supply system for a surgical robot, including an AC to DC converter 101, a power supply interface board 102 and a backup power control board 500. The AC to DC converter 101 is connected to the backup power control board 500. Those skilled in the art can understand that the power supply interface board 102 may be a field programmable gate array (FPGA) component. The DC power control board may be electrically connected to the power supply interface board 102 through a corresponding interface, or may be connected by other means.

Preferably, the power supply interface board 102 may also be provided with several interfaces for directly outputting power supply, such as being connected to a display device, etc. The layout of the interfaces may be uniformly designed in combination with the number of interfaces of the DC power control board and the size of the power supply interface board 102, for example, the interface for directly outputting power supply is arranged on the other side of the DC power control board.

As a preferred embodiment, the power supply system for the surgical robot further includes a first DC power control board 105 and a second DC power control board 106. The first DC power control board 105 and the second DC power control board 106 respectively obtain the 48V DC power and the 24V DC power from the power supply interface board 102 through correspondingly arranged interfaces, and provide the 48V DC power and the 24V DC power to the surgical robot through their own output interfaces.

In this embodiment, two DC power control boards corresponding to different output currents are provided to meet the complicated power supply requirements of the surgical robot. Of course, as mentioned above, the 48V and 24V DC power are just examples herein. According to the needs of the actual environment and different devices, other DC power control boards may be added or output parameters of their corresponding power may be changed.

The power supply system for the surgical robot in this embodiment realizes that the power supply system can work continuously and stably in the scene of unexpected power failure based on providing the backup power supply control board and the power supply interface board.

### Embodiment 3

This embodiment provides a surgical robot. Based on the power supply system in Embodiment 2, the surgical robot can work continuously and stably in the scene of unexpected power failure.

### Embodiment 4

This embodiment further provides a computer-readable storage medium, on which a computer program is stored, and when the program is executed by a processor, the input voltage comparison and selection mechanism as in the above-mentioned Embodiment 1 is realized. The readable storage medium may be a portable disk, a hard disk, a random-access memory, a read-only memory, an erasable programmable read-only memory, an optical storage device, a magnetic storage device or any suitable combination of the above.

In a possible embodiment, the present disclosure may also be realized in the form of a program product, which includes a program code, and when the program product is run on a terminal device, the program code is used to make the terminal device execute the steps in the power supply control method for the surgical robot as described above. The program code for executing the present disclosure may be written in any combination of one or more programming languages, and the program may be completely executed on a user device, partially executed on the user device, executed as an independent software package, partially executed on the user device and partially executed on a remote device, or completely executed on the remote device.

Although specific embodiments of the present disclosure have been described above, those skilled in the art should understand that these are merely illustrative, and the scope of protection of the present disclosure is defined by the appended claims. Those skilled in the art may make many changes or modifications to these embodiments without departing from the principle and substance of the present disclosure, but these changes and modifications all fall within the protection scope of the present disclosure.

1. A power supply protection circuit for a surgical robot, comprising a main power supply module, a backup power supply module and at least one selection circuit, wherein,
the main power supply module and the backup power supply module are located at an input end of the selection circuit and are respectively configured to provide main power and backup power to the selection circuit; and
the selection circuit is configured to determine one of the main power supply module and the backup power supply module as power inputted according to input voltages detected from the main power supply module and the backup power supply module, and output power to supply power to the surgical robot.

2. The power supply protection circuit for the surgical robot according to claim 1, wherein the selection circuit is configured to: select the main power supply module as the power inputted in response to that a first input voltage corresponding to the main power supply module is not less than a preset voltage threshold; and select the backup power supply module as the power inputted in response to that the first input voltage is less than the preset voltage threshold.

3. The power supply protection circuit for the surgical robot according to claim 2, wherein the preset voltage threshold is a maximum output voltage of the backup power supply module.

4. The power supply protection circuit for the surgical robot according to claim 1, wherein the selection circuit is configured to: select the main power supply module as the power inputted in response to that a first input voltage corresponding to the main power supply module is not less than a second input voltage corresponding to the backup power supply module; and select the backup power supply module as the power inputted in response to that the first input voltage is less than the second input voltage.

5. The power supply protection circuit for the surgical robot according to claim 1, wherein the backup power supply module is configured to: output power to the selection circuit in response to that the backup power supply module is used as the power inputted, and store power in response to that the main power supply module is used as the power inputted.

6. The power supply protection circuit for the surgical robot according to claim 5, wherein the backup power supply module comprises a charging device and a rechargeable battery which are electrically connected with each other, and the charging device is configured to store power in the rechargeable battery in response to that the main power supply module is used as the power inputted.

7. The power supply protection circuit for the surgical robot according to claim 6, further comprising a plurality of direct current (DC) to direct current (DC) converter, wherein, input ends of the DC to DC converter are electrically connected to the rechargeable battery, and output ends of the DC to DC converter are electrically connected to a corresponding selection circuit.

8. The power supply protection circuit for the surgical robot according to claim 1, wherein the power supply protection circuit comprises at least two selection circuits with different output voltages.

9. A power supply system for a surgical robot, comprising the power supply protection circuit according to claim 1.

10. A surgical robot, comprising the power supply system according to claim 9.
